SEQUENCE LISTING (2 sequences)

SEQ ID NO: 1 - DNA, 47 bp, synthetic construct
  source 1..47: mol_type = other DNA
ctctcgacgacgtttgcgatgagaaacgtatggtttcgaaggtcgtc

SEQ ID NO: 2 - DNA, 12 bp, synthetic construct
  source 1..12: mol_type = other DNA
agtcgtcgagag

(19) United States
(12) Patent Application Publication
(10) Pub. No.: US 2026/0318905 A1
(43) Pub. Date: Oct. 15, 2026

(54) SWITCH SENSORS AND METHODS OF USE

(71) Applicant: Board of Regents, The University of Texas System, Austin, TX (US)

(72) Inventors: Jing Yong Ye, San Antonio, TX (US); Luis Trabucco, San Antonio, TX (US); Jonathan Shaw, San Antonio, TX (US)

(21) Appl. No.: 19/634,448

(22) Filed: Mar. 31, 2026

Related U.S. Application Data
(60) Provisional application No. 63/780,680, filed on Mar. 31, 2025.

(51) Int. Cl. G01N 33/53; G01N 21/17; G01N 21/64; G01N 33/543; G01N 33/58

(52) U.S. Cl. CPC G01N 33/5308; G01N 21/1702; G01N 21/6428; G01N 33/54373; G01N 33/582

(57) ABSTRACT

Current fluorescent biosensors rely on fluorescent spectrum shifts, limiting the detection technique compatible with the technology. These limitations hinder the ability to utilize fluorescent biosensing technologies in vivo. Switch sensors are biosensors that allow for the sensitive detection of target analytes through a diverse set of detection techniques. Switch sensor monomers are comprised of a platform segment, a linker, and a fluorescent dye monomer. The aptamer or polypeptide-based platform segment drives binding between two switch sensor monomers, ultimately resulting in a fluorescent dye homodimer. The utilization of specific fluorescent dyes allows for a change in the absorption spectrum upon binding of the target analyte. This change in the absorption spectrum broadens the detection capabilities, allowing for photoacoustic applications, absorption spectroscopy, in addition to fluorescence measurements.

### RELATED APPLICATIONS

This application claims priority to U.S. Provisional Patent Application 63/780,680 filed Mar. 31, 2025 which is incorporated herein by reference in its entirety.

### STATEMENT REGARDING FEDERALLY FUNDED RESEARCH

None.

### REFERENCE TO SEQUENCE LISTING

The Sequence Listing, which is a part of the present disclosure, is submitted electronically via EFS-Web as an ASCII formatted sequence listing with a file name of UTSAP0166US.XML, a creation date of Jun. 17, 2026, and a size of 2,823 bytes. The information in the ASCII formatted sequence listing is incorporated herein by reference in its entirety.

### FIELD

Aspects described herein are directed generally to the field of medicine and biochemistry, more particularly the field of detectable probes and bioassays.

### BACKGROUND

Biosensors represent a valuable class of detection systems with significant potential for applications in biomedicine, environmental monitoring, food safety, and beyond. A rapidly evolving subset of biosensors are the aptamer biosensors, attractive in part due to the ease of synthesis as well as their stability. Aptamers are short single-stranded oligonucleotides (DNA or RNA) that possess specificity and affinity for their targets, which can range from small molecules to proteins, ions, cells, and even entire organisms. These attributes make them promising candidates for biosensing technologies, providing a chemically synthesized alternative to antibodies in conventional immunoassays, with advantages including ease of synthesis and stability. Conventional aptamer biosensors function by converting a molecular recognition event-such as the binding of an aptamer to its target-into a measurable signal. These sensors can employ a range of transduction methods, including optical detection.

Fluorescence Resonance Energy Transfer (FRET) is a technique widely used in biosensing applications to monitor molecular interactions, and FRET-based aptamer biosensors have emerged as a useful platform for detecting various targets with high sensitivity and specificity. In FRET, energy transfer occurs between two fluorophores—a donor and an acceptor—when they are in close proximity, typically within 1-10 nm. This non-radiative energy transfer typically quenches donor emission and/or enhances acceptor emission.

Conventional approaches exploiting indocyanine green (ICG) often rely on concentration-dependent self-aggregation into H-type dimers or aggregates, resulting in a blue-shift of the absorption maximum from ~780-789 nm (monomer) to ~715 nm (dimer/H-aggregate). Such aggregation is typically uncontrolled and occurs at high dye concentrations in aqueous media, limiting specificity in complex biological environments. Similarly, while aptamer-based sensors have utilized FRET pairs or intercalating dyes, few exploit controlled proximity of fluorophores to modulate absorption profiles for ratiometric, multi-modal (including photoacoustic) detection. The present invention addresses these limitations by using an analyte-binding domain to precisely control dimer formation/dissociation of fluorescent monomers, enabling specific, sensitive, and versatile analyte detection.

There remains a need for additional detection methods and fluorophore complexes.

### SUMMARY

Embodiments provide novel switch sensors and methods of use that represent an advancement over conventional fluorescent biosensors and aptamer-based detection systems. Certain aspects of the invention use an analyte-binding domain (such as an aptamer, antibody, polypeptide, or molecularly imprinted polymer) operably coupled to a switch sensor domain comprising two fluorescent monomers capable of forming a dimer (e.g., a homodimer). Unlike traditional FRET-based sensors that rely on energy transfer between two different fluorophores and depend primarily on changes in fluorescence emission spectra, the switch sensors described herein exploit a controlled, analyte-induced association or dissociation of fluorescent monomers or analyte-induced dissociation of the dimer. The absorption spectrum is modulated between the dimer and the monomer states. This absorption-based spectral switch enables highly sensitive, ratiometric detection using a broader range of modalities, including absorption spectroscopy, fluorescence spectroscopy and imaging, and photoacoustic spectroscopy and imaging, thereby overcoming limitations associated with fluorescence-only detection in complex biological environments and deep-tissue in vivo applications. The platform further enables detection of enzymatic activity, such as nuclease concentrations, through cleavage-induced separation of the dimer. By precisely controlling dimer formation or dissociation through analyte-binding events, the invention addresses the uncontrolled aggregation issues of prior indocyanine green systems and provides a versatile, stable, and regenerable platform for the specific detection of diverse analytes, including metabolites such as adenosine, lactate, glucose, and ATP, as well as proteins, ions, cells, and other biomolecules. In addition to analyte-binding, the switch sensor platform can be extended to detect enzymatic activity, including nuclease concentrations. In such embodiments, the switch sensor comprises a nucleic acid structure that is susceptible to nuclease-mediated cleavage. Enzymatic digestion of the aptamer or nucleic acid scaffold disrupts the structural integrity of the sensor and results in separation of the fluorescent dye dimer into monomeric units. This cleavage-induced decoupling produces a measurable shift in the absorption spectrum, which can be detected using absorption spectroscopy, fluorescence spectroscopy, and photoacoustic spectroscopy and imaging. Accordingly, the switch sensor enables direct transduction of enzymatic activity into optical and photoacoustic signals, providing a versatile platform for monitoring nuclease activity in addition to molecular recognition events.

Certain embodiments are directed to switch sensors having an analyte-binding domain and a switch sensor domain, e.g., an aptamer switch sensor. The switch sensor domain is formed by two monomers (a monomer being a fluorescent chemical moiety or a fluorescent monomer) that are capable of forming a dimer (e.g., homodimer). The switch sensor detects specific chemical or biological substances at the molecular level. It works by an analyte-binding domain interacting with a target molecule (analyte) and altering/activating the switch sensor domain producing a measurable signal, the measurable signal is the change in absorption of a dimer (e.g., homodimer) to that of the constituent monomers or the change in absorption of constituent monomers, i.e., a switch in absorption from monomer to dimer or dimer to monomer. The analyte-binding domain can be any platform that binds an analyte which alters the structure of the analyte-binding domain upon binding of an analyte resulting in alteration or activation of the switch sensor domain. Analyte-binding domains are molecules or substances or complexes thereof that specifically bind to a target analyte (a substance being measured or detected) with high affinity and selectivity. The analyte-binding domain can include but is not limited to a nanoparticle, non-biologic polymer, or a biologic polymer (polypeptide, polynucleotide, etc.). In particular aspects the analyte-binding domain is an aptamer, an antibody, molecularly imprinted polymers (MIP), and the like. The analyte-binding domain can be made or formed of 1, 2, 3, or more analyte-binding segments (monopartite or multipartite). The analyte-binding segments can be independent (e.g., present on two separate segments that either associate or dissociate upon analyte-binding—for example a double stranded aptamer) or coupled (e.g., both analyte-binding segments are coupled or present as a single analyte-binding segment—for example a single stranded aptamer). The analyte-binding domain can be formed when in contact with a target analyte or the analyte-binding domain can dissociate when binding a target analyte. The one or more analyte-binding segments can be coupled to a fluorescent monomer, each analyte segment can be coupled to a separate monomer. In certain aspects each monomer has the same properties (absorbs and/emits at a particular wavelength profile) or chemical structure. A fluorophore is a molecule or part of a molecule that can absorb light at a specific wavelength and then emit light at a longer wavelength, a process known as fluorescence. The monomers when in proximity, that is forming a dimer (e.g., homodimer), have an absorption property distinct from the monomers when separated. In certain aspects the monomers are within 0, 1, 2, 3, 4, 5, 6, 7, 8, 9, 10, to 11, 12, 13, 14, 15, 16, 17, 18, 19, 20 nm of each other to form a dimer (e.g., homodimer).

In the context of an aptamer sensor, the first analyte-binding segment is 5′ labeled and the second analyte-binding segment is 3′ labeled. One or a combination of the analyte-binding segments forms an analyte-binding domain. Annealing of the analyte-binding segments brings the two fluorescent dye monomers together creating a dimer (e.g., homodimer). The fluorescent dye dimer (e.g., homodimer) results in a shift in the absorption spectrum. Binding of the analyte breaks up the dimer (e.g., homodimer) into the two monomers resulting in an absorption spectrum shift. In certain aspects at least one of the fluorescent dye monomer is indocyanine green. In one aspect the first analyte-binding segment is 3′ labeled and the second analyte-binding segment is 5′ labeled. In one aspect the first and second analyte-binding segments are located on a contiguous polynucleotide. Wherein they can hybridize by the formation of a hairpin loop. In another aspect, the analyte-binding segments are comprised of polynucleotides bound to the fluorescent dye monomers. In one aspect the first and second analyte-binding segments are located on a contiguous polypeptide. Wherein the binding of the peptide allows for a conformation that brings the two fluorescent monomers together. Analytes can be but are not limited to a molecule, a protein, a cell, a metabolite, an ion, or nucleoside. In particular aspects the nucleoside is adenosine.

In one embodiment the distinct wavelength absorption specific to the monomer or dimer (e.g., homodimer) fluorescent dye allows for optical detection methods, including absorption spectroscopy, fluorescence spectroscopy, fluorescence imaging, photoacoustic spectroscopy and photoacoustic imaging.

In another aspect the fluorescent dye dimer (e.g., homodimer) is formed through the two fluorescent dye monomers coming within close proximity to one another.

### Definitions

The following definitions are provided to clarify the meaning of specific terms used throughout this patent application. These terms are defined to ensure a clear and consistent understanding of the invention's scope, embodiments, and claims. Unless otherwise specified, the terms used herein have the meanings set forth below.

As used herein, “Biosensor” refers to an analytical device that integrates a biological component (analyte-binding domain) with a physicochemical detector (switch sensor domain) to detect and measure specific biological or chemical substances. The biological component can be an enzyme, antibody, nucleic acid, receptor, microorganism, or any biological material that recognizes the target analyte with high specificity. This component's interaction with the target substance initiates a biochemical reaction or binding event. The physicochemical detector, after the biological recognition, converts the biological response into an electrical, optical, or other forms of measurable signal.

“Polypeptide” refers to any peptide or protein comprising amino acids joined by peptide bonds or modified peptide bonds.

“Aptamer” refers to a polynucleotide (e.g., DNA or RNA) molecule(s) that can bind to specific target molecules with high affinity and specificity. Aptamers fold into unique three-dimensional shapes due to their sequence, which allows them to recognize and bind to targets like proteins, small molecules, or even whole cells. An aptamer can comprise one or more polynucleotides, including but not limited to DNA or RNA molecule(s), or hybrid polynucleotides that can bind to specific target molecules with high affinity and specificity.

“Monomer” refers to a molecule that can react with other molecules of the same or different types to form a polymer (e.g., a dimer). A fluorescent dye monomer is a molecule that can be brought in proximity (0 to 20 nm) of another monomer to form a dimer (e.g., homodimer).

“Homodimer” refers to a molecule or molecular complex consisting of two identical or similar subunits called monomers that are within proximity of each other. In chemistry, dimers often form when two molecules of the same compound come together, either through covalent bonds, hydrogen bonds, or other types of interactions. Dimers are significant because the association can alter the chemical or physical properties compared to the individual monomers.

“Dimer” refers to a molecular complex consisting of two subunits, referred to as monomers, that are brought into close proximity (typically 0 to 20 nm). In the context of the present switch sensors, a dimer is a homodimer or a heterodimer formed by two fluorescent dye monomers. The dimer may form through non-covalent interactions, hydrophobic effects, π-π stacking, or other attractive forces between the fluorescent monomers when they are held in proximity by the analyte-binding domain. Formation or dissociation of the dimer results in a distinct change in the absorption spectrum (and potentially fluorescence or photoacoustic properties) compared to the separated individual monomers. This spectral switch enables the detection of analyte-binding events.

“Fluorescent dye” or “fluorescent monomers” refers to compounds that absorb light at a specific wavelength and then emit light at a longer wavelength, a process known as fluorescence. They have specific excitation wavelengths (where they absorb light) and emission wavelengths (where they emit light). For one-photon excitation, the emission wavelength is longer than the excitation wavelength, which corresponds to the Stokes shift. For two-photon excitation, the emission wavelength is shorter than the excitation wavelength.

“Linker” refers to a molecular component that connects different parts of a biosensor system. They help in maintaining the correct orientation and distance of the biological recognition element from the sensor surface, which is crucial for effective binding and signal transduction. They often provide flexibility, allowing the recognition element to interact freely with its target analyte. Linkers can enhance the stability of the biosensor by preventing the denaturation or degradation of the recognition element.

“Absorption spectrum” refers to the spectrum of electromagnetic radiation (light) that has been absorbed by a moiety.

The phrase “specifically binds” to a target refers to a binding reaction that is determinative of the presence of the molecule in the presence of a heterogeneous population of other biologics.

“Analyte” refers to a substance or chemical constituent that is of interest in an analytical procedure. In the context of chemistry, biology, or medical science, it's the specific component within a sample that is being measured or analyzed.

“Metabolite” refers to a small molecule or intermediate product of metabolism within a biological system. Metabolites are a species of analyte that can be specifically detected by the switch sensors described herein when the analyte-binding domain is configured to recognize and bind said metabolite with high affinity and selectivity. Metabolites include, but are not limited to, nucleosides (such as adenosine), organic acids (such as lactate), sugars (such as glucose), amino acids (such as glutamine, tyrosine, alanine, or glutamate), nucleotides and their derivatives (such as ATP), neurotransmitters (such as dopamine or serotonin), lipids and lipid-derived molecules (such as cholesterol or β-hydroxybutyrate), and other biochemical compounds produced or consumed during cellular metabolic processes, including those involved in glycolysis, the tricarboxylic acid cycle, energy transfer, and signaling pathways. In particular aspects, the metabolite may be adenosine, lactate, glucose, or ATP. Detection of such metabolites enables applications in medical diagnostics, monitoring of metabolic disorders, and in vivo or environmental sensing via changes in the absorption, fluorescence, or photoacoustic profiles of the switch sensor domain.

“Absorption spectroscopy” refers to a technique used in analytical chemistry to determine the presence and concentration of a chemical substance by measuring how much light of a particular wavelength is absorbed by a sample.

“Fluorescence spectroscopy” refers to a type of spectroscopy that involves the measurement of the intensity and wavelength distribution of light emitted from a sample after it has absorbed electromagnetic radiation, typically in the ultraviolet or visible spectrum.

“Fluorescence imaging” refers to a technique which involves the use of a light source to excite fluorescent molecules (known as fluorophores) within a sample or tissue. These fluorophores absorb light at a specific wavelength, then emit light at a longer wavelength. This emitted light, or fluorescence, is captured and used to create images that can reveal the location, concentration, or interaction of the fluorophores within the sample or tissue.

“Photoacoustic spectroscopy” (PAS) refers to an analytical technique used to measure the absorption of light by a sample or tissue, based on the photoacoustic effect. Photoacoustic spectroscopy combines aspects of both optical and acoustic methods, offering a unique tool for spectroscopic analysis where traditional methods might face limitations.

“Photoacoustic imaging” (PAI) refers to a biomedical imaging technique that combines the advantages of both optical and ultrasound imaging to provide high-resolution, deep-tissue imaging.

“Polynucleotide,” synonymously referred to as “nucleic acid molecule” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, double-stranded, or a mixture of single- and double-stranded regions.

A biomarker is an organic biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio.

“Assay” refers to a biochemical or chemical procedure used to test or measure the presence, amount, or functional activity of a target entity (such as a molecule, compound, or biological substance). Assays are fundamental tools in scientific research, particularly in fields like biology, chemistry, medicine, and pharmacology.

These definitions are intended to be read in their broadest reasonable interpretation consistent with the specification and are not limited to any specific materials, circuit topologies, numerical values of n, or biological examples disclosed herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1B. Schematic illustration of exemplary aptamer-based switch sensors. (A) Two complementary aptamer segments (CTCTCGACGACGTTTGCGATGAGAAACGTA TGGTTTCGAAGGTCGTC (SEQ ID NO:1) and AGTCGTCGAGAG (SEQ ID NO:2)) labeled at opposing termini with ICG, forming a homodimer upon hybridization. (B) Absorption spectra showing the shift upon homodimer formation.

FIG. 2. Illustration of one working principle where each of the terminals of an aptamer is labeled with an ICG molecule. When a hairpin is formed, it brings the two ICG molecules together to form a dimer bringing the two ICG molecules into proximity to form a homodimer with altered absorption.

FIG. 3A-3B. Illustration of an aptamer switch sensor for Adenosine. (A) Illustration for aptamer switch sensor (aptamer 1 (SEQ ID NO:1) and aptamer 2 (SEQ ID NO:2) binding adenosine. (B) Absorbance spectrum of aptamer switch sensor binding adenosine.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment and not intended to imply that the scope of the disclosure, including the claims, is limited to that embodiment.

Provided herein, are switch sensors, that in several embodiments allow for the sensitive and specific detection of analytes, measurable by techniques including photoacoustic imaging.

The switch sensor, described herein, is comprised of an analyte-binding domain coupled to a first and second switch monomer, together resulting in a fluorescent biosensor. Switch sensors include an analyte-binding segment, a linker, and a switch sensor composed of two or more fluorescent molecules referred to as a fluorescent monomer. Each component is described in detail below. The analyte-binding segments through a binding or annealing event bring the first and second switch monomers together or disassociate the switch monomers. In certain aspects a fluorescent monomer can be coupled to a linker connecting the analyte-binding domain segment to the fluorescent monomer. In certain embodiments identical fluorescent dye monomers are used as the first and second fluorescent monomers. In certain aspects, upon the binding or annealing of the analyte-binding segments coupled to fluorescent monomers, the fluorescent monomers are brought together to create a fluorescent dimer (e.g., homodimer). As the two fluorescent monomers form a dimer (e.g., homodimer), either through binding or coming within proximity (0 to 20 nm) to one another, there is a modulation of the absorption spectrum resulting in a measurable distinction when the first and second monomers come together. The analyte-binding domain allows for the specific binding of a target analyte.

In certain aspects upon binding the analyte through either a steric hindrance or a conformational change a distancing event takes place separating the fluorescent monomers, i.e., separating the fluorescent dimer (e.g., homodimer) into two fluorescent monomers. The separation causing the absorption spectrum to shift from that of the fluorescent dimer (e.g., homodimer) to that of the fluorescent monomer allowing for measurable detection of the target analyte. The mechanism can work in the opposite direction as well where binding an analyte brings two fluorescent monomers in proximity. Modulation of the absorption spectrum of the switch sensor, opens up the possibility for operating uniquely and synergistically with different optical detection methods, including absorption spectroscopy, fluorescence spectroscopy and imaging, and photoacoustic spectroscopy and imaging.

Switch sensor fluorescent dyes have unique characteristics comprising an absorption spectrum of fluorescent monomer that is distinct from an absorption spectrum of the dimer (e.g., homodimer). Absorption spectra are characteristic curves showing how much light a dye absorbs at different wavelengths. The changes in absorption increase the possibilities for detection techniques over current fluorescent biosensors, including absorption spectroscopy, fluorescence spectroscopy and imaging, and photoacoustic spectroscopy and imaging.

In certain embodiments, switch sensor fluorescent dyes have unique characteristics comprising an emission spectrum of fluorescent monomer that is distinct from an emission spectrum of the dimer (e.g., homodimer). An emission spectrum is the pattern of specific wavelengths of light emitted by an atom, molecule, or substance when its electrons transition from higher to lower energy states after being excited by energy, such as heat or electricity.

The linker between a binding domain and a fluorescent monomer can maintain the functionality of the binding domain while allowing the fluorescent monomer to effectively report the binding event. If the binding domain is an aptamer the linker can be comprised of a chemical moiety such as an alkyl chain, polyethylene glycol (PEG), or other flexible molecules. If the binding domain is a polypeptide the linker is chosen based on reactive groups available on the polypeptide some examples being N-hydroxysuccinimide esters or maleimides. The linker is to provide enough spatial separation between the binding platform and the fluorescent monomer to prevent steric hindrance, which might affect the folding or binding capabilities of the platform segment.

Binding domain segments can include polymers, aptamers, or polypeptides that fold into complex secondary and tertiary structures, often involving loops, bulges, and stems. The binding domain is typically part of this structure, where specific polymers, nucleotides, or amino acids are crucial for recognizing and binding to the analyte. There are broad possibilities for what the analyte can be, including cells, proteins, ions, and metabolites. In the design of the switch sensor the binding of the analyte can create an association or dissociation between binding domain components, ultimately resulting in a shortening or distancing event between the fluorescent monomers.

In one aspect the switch sensor is comprised of polypeptide binding domain. Wherein the binding domain is comprised of an unbranched chain of amino acids linked by peptide bonds. The ability of the polypeptide to form specific three-dimensional structures that interact selectively with other molecules allows it to serve as the binding domain of the switch sensor. Polypeptides are inherently biocompatible, meaning they are less likely to cause adverse reactions when used in biological environments or in vivo applications. This is particularly important for medical or diagnostic biosensors that might interact with human tissues or fluids. The use of polypeptides in biosensors leverages their biochemical properties to achieve high specificity, sensitivity, and functional versatility.

In another aspect the switch sensor can be comprised of aptamer binding domains. The following description is not limited to the aptamer form of the switch sensor but is used to describe the process of the switch sensor as well as define differences with currently used biosensor technologies. Aptamers offer several benefits over traditional biomolecular recognition elements (e.g., antibodies and the like). An aptamer can provide high affinity and high specificity binding to a target, thus reducing cross-reactivity and increasing detection accuracy. Unlike proteins, aptamers are chemically synthesized and more resistant to denaturation or degradation, making them highly stable over a wide range of environmental conditions, including high temperature and pH variations. The ability to regenerate aptamers through chemical treatment allows for multiple uses in sensor systems, reducing cost in long-term applications. Furthermore, aptamers can be easily modified with functional groups, fluorescent tags, or nanomaterials to enhance detection and signal transduction, making them adaptable to various biosensor platforms.

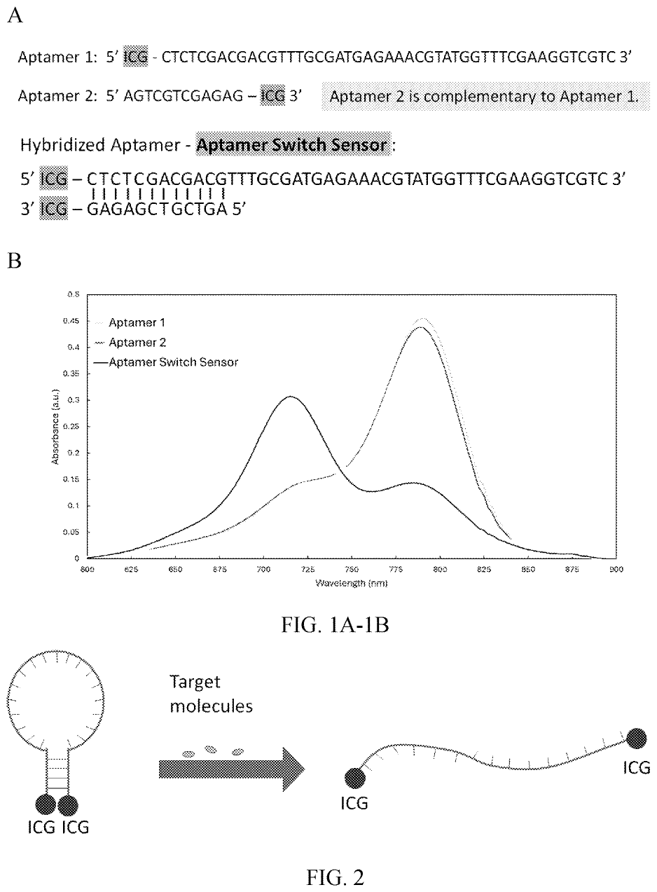

The aptamer switch sensor described herein has a unique working mechanism as compared to the traditional FRET-based aptamer biosensors. The design of the aptamer switch sensor is illustrated in FIG. 1A-1B. In this example each aptamer switch sensor employs two indocyanine green (ICG) molecules, which form a homodimer either through the hybridization process of two complementary aptamers or through a hairpin formation. Other dye molecules with a property similar to ICG may also be used, if their absorption spectra of a monomer and a homodimer are different. The absorption spectra of the ICG labeled aptamers are altered when the ICG homodimer is formed due to the unique feature of ICG (FIG. 2). The hybridized aptamer with an ICG homodimer can then serve as a novel biosensor for a wide range of applications.

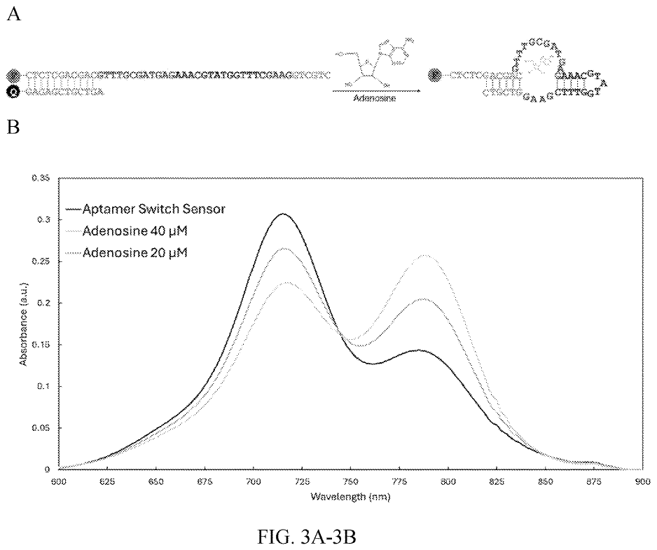

Upon the interaction of an aptamer switch sensor with a specific target analyte, the ICG homodimer is separated into two monomers due to the replacement of one of the complementary aptamers with an analyte or due to the restructuring of the hairpin structure. The separation of the ICG homodimer results in the modulation of the absorption spectrum of the aptamer switch sensor, thus allowing ratiometric optical sensing for highly accurate quantification of the target molecules or cells. Ratiometric refers to a measurement technique or system where the measurement is based on the ratio of two quantities rather than on an absolute value. This sensor is distinct from conventional molecular beacons or a FRET agent that contains two different fluorophores and rely on fluorescence spectrum shifts. In contrast, the absorption spectrum of the aptamer switch sensor is modulated, which opens up the possibility for operating uniquely and synergistically with different optical detection methods. Thus, this unique aptamer switch sensor may facilitate efficient and accurate ratiometric quantification of target molecules in vitro and in vivo. An example of detection of adenosine is shown in FIG. 3. When one of the complementary aptamers is designed to have a high binding affinity with adenosine, the spectra of the aptamer switch sensor sensitively changes with the concentration of adenosine.

The versatility of the switch sensor regarding the platform segment allows for diverse analyte potential. Prospective analytes include cells, proteins, ions, and metabolites. With complexities ranging from small molecules to entire organisms. This adaptability makes switch sensors applicable in fields like medical diagnostics, environmental monitoring, food safety, and drug development.

### I. Switch Sensor

A switch sensor has an analyte-binding domain and a switch sensor domain, e.g., an aptamer coupled with a switch sensor. The switch sensor domain is formed by two monomers (a monomer being a fluorescent chemical moiety or a fluorescent monomer) forming a dimer (e.g., homodimer). The switch sensor detects specific chemical or biological substances at the molecular level. It works by an analyte-binding domain interacting with a target molecule (analyte) and altering/activating the switch sensor domain producing a measurable signal, the measurable signal is the change in absorption of a dimer (e.g., homodimer) to that of the constituent monomers or the change in absorption of constituent monomers to that of the dimer (e.g., homodimer), i.e., a switch in absorption from monomer to dimer (e.g., homodimer) or dimer (e.g., homodimer) to monomer. The analyte-binding domain can be any platform that binds an analyte which alters the structure of the analyte-binding domain upon binding of an analyte resulting in alteration or activation of the switch sensor domain. Analyte-binding domains are molecules or substances or complexes thereof that specifically bind to a target analyte (a substance being measured or detected) with high affinity and selectivity. The analyte-binding domain can include but is not limited to a nanoparticle, non-biologic polymer, or a biologic polymer (polypeptide, polynucleotide, etc.). In particular aspects the analyte-binding domain is an aptamer, an antibody, a receptor, molecularly imprinted polymers (MIP), and the like. The analyte-binding domain can be made or formed of 1, 2, 3, or more analyte-binding segments (monopartite or multipartite). The analyte-binding segments can be independent (e.g., present on two separate segments that either associate or dissociate upon analyte-binding e.g., a double stranded aptamer)) or coupled (e.g., both analyte-binding segments are covalently coupled or present as a single analyte-binding segment (e.g., a single stranded aptamer)). The analyte-binding domain can be formed when in contact with a target analyte or the analyte-binding domain can dissociate when binding a target analyte. The one or more analyte-binding segments can be coupled to a fluorescent monomer, each analyte segment can be coupled to a separate monomer. In certain aspects each monomer has the same properties (absorbs and/emits at a particular wavelength profile) or chemical structure. A fluorophore is a molecule or part of a molecule that can absorb light at a specific wavelength and then emit light at a longer wavelength, a process known as fluorescence. The monomers when in proximity, that is forming a dimer (e.g., homodimer), have a fluorescent property distinct from the monomers when separated.

In embodiments where the analyte-binding domain comprises a polypeptide, the domain is designed as a single-chain or multi-domain peptide that undergoes a conformational change upon analyte-binding. For example, a polypeptide segment can be engineered with flexible linkers (e.g., (Gly-Ser), repeats) connecting two regions that fold to bring the attached fluorescent monomers into proximity (0-20 nm) in the absence of analyte, forming the dimer (e.g., homodimer). Upon analyte-binding (e.g., to a specific epitope or allosteric site), steric hindrance or unfolding separates the monomers, shifting the absorption spectrum from dimer (e.g., homodimer)-dominant (blue-shifted, e.g., ~700 nm for ICG H-type dimer) to monomer-dominant (~780 nm). Conjugation of fluorescent monomers (e.g., ICG) to the polypeptide can be achieved via maleimide-thiol chemistry: Introduce a cysteine residue at the N- or C-terminus of each binding segment, react with ICG-maleimide in PBS (pH 7.2) at room temperature for 2-4 hours, followed by purification via size-exclusion chromatography. Prophetic example: A calmodulin-based polypeptide domain binds Ca²⁺ ions, inducing a conformational shift that dissociates attached ICG monomers; expected ratiometric change in absorbance at 700 nm vs. 780 nm enables ion detection in biological fluids.

### A. Aptamers

Aptamers are short, single-stranded DNA, RNA, or peptide molecules that bind to specific molecular targets, such as proteins, with high affinity and specificity. They are often compared to antibodies due to their targeting abilities but are smaller in size and can be chemically synthesized, offering advantages in stability and production.

Aptamers fold into unique 3D shapes that allow precise binding to targets. Aptamers can be used in but are not limited to diagnostics (e.g., detecting biomarkers), therapeutics (e.g., targeting disease-related proteins), research tools (e.g., studying molecular interactions) and the like. Aptamers typically have high specificity and affinity, and low immunogenicity (less likely to trigger immune responses). Aptamers can be selected through a process called SELEX (Systematic Evolution of Ligands by Exponential Enrichment) and are increasingly explored for medical and biotechnological innovations.

The aptamer-switch biosensor described herein has a unique working mechanism and is distinctly different from the traditional FRET-based aptamer biosensors. The design of the aptamer-switch biosensor is shown in FIG. 1. In this example each aptamer sensor employs two indocyanine green (ICG) molecules, which form a dimer either through the hybridization process of two complementary aptamers or through a hairpin formation. Other dye molecules with a property similar to ICG may also be used, if their absorption spectra of a monomer and a dimer are different. The absorption spectra of the ICG labeled aptamers are altered when the ICG dimer is formed due to the unique feature of ICG (FIG. 2). The hybridized aptamer with an ICG dimer can then serve as a novel biosensor for a wide range of applications in biomedicine, environmental monitoring, and food safety inspection by specific interaction with different targets (including small molecules, proteins, ions, and cells).

Upon the interaction of an aptamer-switch biosensor with a specific target molecule, the ICG dimer is separated into two monomers due to the replacement of one of the complementary aptamers with a target molecule or due to the separation of the hairpin structure. The separation of the ICG dimer results in the modulation of the absorption spectrum of the aptamer-switch biosensor, thus allowing measurements with absorption spectroscopy, fluorescence spectroscopy and imaging, and photoacoustic spectroscopy and imaging, as well as ratiometric optical sensing for highly accurate quantification of the target molecules or cells. This sensor is distinct from conventional molecular beacons or a FRET agent that contains two different fluorophores and rely on fluorescence spectrum shifts. In contrast, the absorption spectrum of the aptamer-switch biosensor is modulated, which opens up the possibility for operating uniquely and synergistically with different optical detection methods, including absorption spectroscopy, fluorescence spectroscopy and imaging, and photoacoustic spectroscopy and imaging. Thus, this unique aptamer-switch biosensor may facilitate efficient and accurate ratiometric quantification of target molecules in vitro and in vivo. An example of detection of adenosine is shown in FIG. 3. When one of the complementary aptamers is designed to have a high binding affinity with adenosine, the spectra of the aptamer-switch sensor sensitively changes with the concentration of adenosine.

A new aptamer biosensor that uniquely changes its absorption spectrum when interacting with a target has been developed. As the absorption spectrum is a more fundamental property than fluorescence, this new type of sensor allows for higher flexibility in detection modalities, making it useful in applications where traditional FRET-based aptamer sensors fall short. This distinction broadens the biosensor's applicability, potentially extending its utility into more challenging diagnostic scenarios or environmental monitoring where typical FRET systems might face limitations due to their dependence on specific fluorescent properties.

### B. Antibodies

Antibodies are proteins produced by the immune system that can specifically bind to target molecules, known as analytes or antigens. They are widely used in detection methods due to their high specificity and affinity for these targets. Antibodies recognize and bind to specific regions (epitopes) on the analyte. This binding is highly selective, making antibodies ideal for detecting analytes in complex samples (e.g., blood, saliva, or environmental samples).

In one example, antibodies labeled with a monomer can be immobilized on a surface (e.g., a plate). The sample containing the analyte is added, and the analyte binds to the antibody. In certain aspects a secondary antibody labeled with a second monomer binds to the analyte and the signal before and after addition of sample and/or secondary antibody can be determined.

Typically monoclonal antibodies are used because they are highly specific, targeting a single epitope on the analyte.

For antibody-based switch sensors, the analyte-binding domain comprises a monoclonal or polyclonal antibody (or Fab fragment) specific to the target (e.g., protein biomarker). In a preferred sandwich format, a capture antibody is immobilized on a surface (e.g., microtiter plate or nanoparticle), and a detection antibody is conjugated to one fluorescent monomer. A secondary linker antibody or streptavidin-biotin system brings a second identical monomer in proximity when analyte bridges the two antibodies, forming the dimer (e.g., homodimer). Analyte absence keeps monomers separated. Conjugation uses NHS-ester chemistry: React amine groups on lysine residues of the antibody with ICG-NHS in carbonate buffer (pH 8.5-9.0) for 1 hour, quench with glycine, and purify by dialysis or column chromatography. Prophetic example: Anti-PSA antibody pair for prostate-specific antigen detection; homodimer formation upon sandwich assembly yields blue-shifted absorption (~700 nm peak dominant), with dissociation (or lack of bridging) restoring monomer spectrum. This enables ratiometric detection in serum samples.

### C. Molecularly Imprinted Polymers (MIP)

Molecularly imprinted polymers (MIPs) are synthetic materials designed to mimic biological recognition systems, such as antibodies, for detecting specific analytes. They are created by polymerizing functional monomers around a target analyte (template), which is later removed, leaving behind cavities that are complementary in size, shape, and chemical functionality to the analyte. MIPs act as artificial receptors with high selectivity for the target analyte due to the specific cavities formed during synthesis. MIPs can distinguish the target analyte from structurally similar compounds, making them ideal for complex sample matrices (e.g., biological fluids, environmental samples).

MIP-based embodiments use synthetic polymers imprinted with the analyte template during polymerization. Functional monomers (e.g., methacrylic acid, acrylamide) form complementary cavities; after template removal, rebinding induces conformational swelling or local restructuring that alters monomer proximity. Fluorescent monomers (e.g., ICG) are covalently incorporated during synthesis (e.g., via co-polymerization with ICG-acrylate derivative) or post-synthetically attached to surface-exposed groups. Prophetic synthesis: Mix template analyte (e.g., small molecule toxin), functional monomer, cross-linker (ethylene glycol dimethacrylate), and initiator in porogen solvent; add ICG-methacrylate conjugate; polymerize under UV or heat, grind/wash to remove template. Rebinding causes cavity closure, bringing proximal ICG units into homodimer proximity for absorption shift. This format suits environmental monitoring in complex matrices.

### Other Binding Domains (Nanoparticles, Receptors, Non-Biologic Polymers)

Nanoparticle-based domains (e.g., gold or silica NPs functionalized with analyte receptors) position monomers on the surface; analyte-induced aggregation or conformational change modulates proximity. Receptors (e.g., G-protein coupled) follow similar polypeptide principles. Non-biologic polymers use analyte-responsive swelling/shrinking to control monomer distance. Conjugation adapts chemistries above (e.g., thiol-gold for NPs).

### D. Use of Switch Sensors

Switch sensors as described herein can be used in analyte detection assays. These detection assays are laboratory techniques designed to identify and quantify specific substances (analytes) in a sample. These assays are widely used in fields such as medical diagnostics, environmental monitoring, and research to detect molecules like proteins, nucleic acids, small molecules, or ions. A sample is the material containing the analyte (e.g., blood, urine, water, or tissue extract). The target analyte is the specific substance to be detected (e.g., glucose, DNA, adenosine, etc.). A binding domain of the switch sensor is the moiety that selectively binds to or interacts with the analyte. The switch sensor domain generates the measurable output produced upon analyte interaction.

Photoacoustic detection is a technique that uses light and sound to detect and analyze materials. A sample is exposed to a pulsed or modulated light source, typically a laser. The light is absorbed by the sample or an activated switch sensor, causing localized heating. The absorbed energy leads to a rapid temperature increase, causing the sample to expand and generate pressure waves (sound waves). These sound waves, or acoustic signals, are detected by sensitive microphones or transducers. The detected signals are analyzed to provide information about the sample's composition, concentration, or other properties.

### Fluorescent Monomers Beyond ICG & Spectral Details

While ICG is preferred (monomer absorption peak ~780-789 nm; homodimer/H-aggregate blue-shifted to ~715 nm), other cyanine dyes exhibiting concentration- or proximity-dependent spectral shifts (monomer vs. homodimer/aggregate) may be used if monomer and homodimer profiles differ sufficiently for detection. For ICG, homodimer formation typically results in a blue-shift in absorption maximum, enabling ratiometric measurements (e.g., absorbance ratio at 715 nm/780 nm increases with homodimer fraction).

Photoacoustic applications leverage the absorption shift: Monomer (~780 nm) and homodimer (~715 nm) generate distinct PA signals due to differential light-to-sound conversion. In tissue phantoms or in vivo, pulsed laser excitation at multiple wavelengths allows ratiometric PA imaging (e.g., PA715/PA780 ratio reports analyte concentration). Fluorescence mode monitors changes in emission intensity or quantum yield if homodimer quenches (common in H-aggregates). Prophetic protocol: Excite at 780 nm (monomer-dominant) and 715 nm (homodimer-dominant); detect ultrasound waves via transducer for deep-tissue analyte mapping.

### II. Switch Sensor Kits

In some embodiments, a kit is provided for detecting one or more analytes in a sample using a switch sensor described herein. The kit may include components and reagents configured to facilitate accurate and efficient analyte detection. The kit may comprise one or more of a (i) switch sensor configured to detect the presence or concentration of one or more analytes in a sample, the switch sensor comprising an analyte-binding domain responsive to the analyte, wherein the switch sensor domain undergoes a conformational or functional change upon interaction with the analyte generating a detectable signal based on the change. (ii) One or more devices for collecting and preparing a sample for analysis, such as a swab, pipette, syringe, or sample container. (iii) One or more reagents for preparing the sample or facilitating the interaction between the analyte and the switch sensor, including but not limited to: Buffers for maintaining pH or ionic strength; Stabilizing agents for preserving analyte integrity; Calibration standards for calibrating the switch sensor; and Control samples containing known concentrations of the analyte for validating sensor performance. (iv) A set of instructions, in printed or electronic form, describing for example procedures for preparing the sample; methods for applying the sample to the switch sensor; interpretation of the detectable signal; and guidelines for calibration, validation, and troubleshooting.

The kit may further include one or more of the following: (i) a reader device or adapter for quantifying or interpreting the detectable signal generated by the switch sensor; (ii) disposable or reusable components, such as cartridges or cassettes; data storage or transmission tools for recording or sharing results; and personal protective equipment, such as gloves or masks, for safe handling of samples and reagents.

The kit can be designed for use in various settings, including but not limited to clinical laboratories, point-of-care facilities, research laboratories, or at-home testing environments. The components of the kit may be packaged in a single container or in separate containers, depending on storage requirements and ease of use.

### III. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 Detection of Adenosine

In this working example, an aptamer switch sensor comprising SEQ ID NO: 1 and SEQ ID NO: 2 labeled with ICG was prepared. Upon titration with increasing concentrations of adenosine, a concentration-dependent shift in absorbance was observed at wavelengths corresponding to the homodimer (~715 nm) and monomer (~780 nm) forms of ICG, confirming analyte detection. The fluorescent dye indocyanine green was utilized, it was bound to complimentary aptamers, one of which contained a platform segment with a high binding affinity for adenosine. In FIG. 3 upon the addition of increasing concentrations of adenosine there was a shift in the absorbance at the distinct wavelengths corresponding to the homodimer and monomer versions of the dye showing binding of the target adenosine in a concentration dependent manner.

1. A switch sensor for detecting a target analyte, comprising:
an analyte-binding domain configured to specifically bind to the target analyte, wherein binding of the target analyte results in a structural alteration of the analyte-binding domain; and
a switch sensor domain comprising a first and a second fluorescent monomers forming a dimer when either a target analyte is present or when a target analyte is absent, wherein the dimer exhibits a first profile when the first and second monomers are in proximity of each other, and the monomers exhibit a second profile distinct from the first profile when the first and second monomers are separated,
wherein alteration of the analyte-binding domain structure upon binding the target analyte results in a measurable change in from the first profile to the second profile or from the second profile to the first profile.

2. The switch sensor of claim 1, wherein the first and/or second profile is an absorption spectra profile of the dimer or monomers.

3. The switch sensor of claim 1, wherein the first and/or second profiles is a fluorescence emission spectrum, intensity, or quantum yield of the dimer or monomers.

4. The switch sensor of claim 1, wherein the first and/or second profiles is a photoacoustic signal intensity, wavelength dependence, or amplitude of the dimer or monomers.

5. The switch sensor of claim 1, wherein the analyte-binding domain comprises one or more analyte-binding segments configured to associate or dissociate upon binding the target analyte.

6. The switch sensor of claim 5, wherein the analyte-binding domain comprises a single analyte-binding segment forming a monopartite structure.

7. The switch sensor of claim 5, wherein the analyte-binding domain comprises two or more analyte-binding segments forming a multipartite structure, wherein the analyte-binding segments are configured to associate or dissociate upon binding the target analyte.

8. The switch sensor of claim 5, wherein at least one analyte-binding segment is coupled to at least one of the fluorescent monomers.

9. The switch sensor of claim 1, wherein the analyte-binding domain is an aptamer, an antibody, a receptor, or a molecularly imprinted polymer (MIP).

10. The switch sensor of claim 1, wherein each fluorescent monomer has a similar chemical structure and optical properties.

11. The switch sensor of claim 1, wherein the measurable change is a shift in absorption wavelength, a change in absorption strength, or both, resulting from a transition between the dimer and the separated monomers or between the separated monomers and the dimer.

12. The switch sensor of claim 1, wherein the measurable change is a shift in fluorescence emission wavelength, intensity, or both, resulting from a transition between the dimer and the separated monomers or between the separated monomers and the dimer.

13. The switch sensor of claim 1, wherein the measurable change is a wavelength shift dependence of photoacoustic signal generation, photoacoustic signal intensity, or both, resulting from a transition between the dimer and the separated monomers or between the separated monomers and the dimer.

14. The switch sensor of claim 1, wherein the analyte-binding domain is configured to form or dissociate upon binding the target analyte, thereby altering the proximity of the fluorescent monomers in the switch sensor domain.

15. A method for detecting a target analyte, comprising:
contacting a sample containing or suspected of containing the target analyte with a switch sensor comprising: (i) an analyte-binding domain configured to specifically bind to the target analyte with high affinity and selectivity, wherein binding of the target analyte structurally alters the analyte-binding domain; and (ii) a switch sensor domain comprising two fluorescent monomers forming a dimer, wherein the dimer exhibits a first profile when the monomers are in proximity, and the monomers exhibit a second profile distinct from the first fluorescence profile when separated; and
detecting a measurable change in absorption, or fluorescence, or photoacoustic signals from the first profile to the second profile or from the second profile to the first profile, wherein the change indicates target analyte presence.

16. The method of claim 15, wherein the analyte-binding domain comprises one or more analyte-binding segments, and binding of the target analyte causes the analyte-binding segments to associate or dissociate, altering the proximity of the fluorescent monomers.

17. The method of claim 15, wherein the analyte-binding domain is selected from the group consisting of an aptamer, an antibody, a receptor, and a molecularly imprinted polymer (MIP).

18. The method of claim 15, wherein each fluorescent monomer has a similar chemical structure and optical properties, and the measurable change is in absorption, or fluorescence emission, or photoacoustic signals, or all.

19. An aptamer switch sensor comprising a first aptamer segment with a 5′ or 3′ label comprising a first fluorescent monomer having a first fluorescent wavelength absorption and a second aptamer segment with a 3′ or 5′ label having a second fluorescent monomer also having the first fluorescent wavelength absorption, wherein the first and second aptamer segments anneal when the aptamer is not engaged with an analyte and the 5′ label and 3′ label form a dimer having a second absorption spectra that is distinct from that of the fluorescent monomer.

20. The sensor of claim 19, wherein the first aptamer segment and second aptamer segment are a polynucleotide.

21.-42. (canceled)
